(19) United States
(12) Patent Application Publication
(10) Pub. No.: US 2023/0287483 A1
(43) Pub. Date: Sep. 14, 2023

(54) METHOD OF IDENTIFYING A PLURALITY OF MICROORGANISMS AND BIOMARKERS FOR ANTIBIOTIC RESISTANCE FROM A SAMPLE USING NUCLEICACID AMPLIFICATION TECHNOLOGY (NAT)

(71) Applicants: MANI M, Coimbatore (IN); MICROBIOLOGICAL LABORATORY, Coimbatore (IN)

(72) Inventors: Mani M, Coimbatore (IN); Brahmadathan K.N, Coimbatore (IN); Helen Hencida.T, Coimbatore (IN)

(21) Appl. No.: 18/119,750

(22) Filed: Mar. 9, 2023

(30) Foreign Application Priority Data
Mar. 9, 2022 (IN) 202241012897

(51) Int. Cl. C12Q 1/6851; C12Q 1/686; C12Q 1/689; G16B 40/10

(52) U.S. Cl. CPC C12Q 1/6851; C12Q 1/686; C12Q 1/689; G16B 40/10; C12Q 2600/16

(57) ABSTRACT

A method of identifying a plurality of microorganisms and their biomarkers for antibiotic resistance from a sample using Nucleic Acid amplification Technology (NAT) is provided. The method includes (i) extracting nucleic acid from a sample; (ii) preparing molecular biomarkers comprising a forward primer and a reverse primer for target genes of each of the plurality of microorganisms as a master mix; (iii) adding the extracted nucleic acid into the master mix to obtain a reaction mixture; (iv) performing a polymerase chain reaction (PCR) on the reaction mixture; (v) identifying a melt temperature for the amplicons; (vi) generating a melt curve for each target gene based on the identified melt temperatures and an amplification curve after the PCR amplification; and (vii) determining, using a Melt Curve analysis technique, Ct values, endpoint fluorescence level and melting profiles of the target genes by analyzing the amplification and melt curves to identify the plurality of microorganism present in the sample.

### BACKGROUND

### Technical Field

The embodiments herein generally relate to identifying a plurality of microorganisms and biomarkers for antibiotic resistance from a clinical sample, more particularly to a method of identifying one or more microorganisms from a sample using Nucleic Acid amplification Technology (NAT).

### Description of the Related Art

Rapid and accurate detection of pathogens from a clinical sample is unidentifiable by conventional microbiological methods and remains a significant challenge in the treatment of patients with fatal clinical conditions such as suspected sepsis. The latter is a life-threatening clinical condition resulting primarily from bacterial infection and less frequently from fungal and/or viral infection. Sepsis-related deaths are frequent among critically ill patients admitted to intensive care units (ICUs). Also, neonates (infants less than four weeks old) are highly prone to sepsis due to their deficiency in an adaptive immune response.

Predominant bacterial species causing sepsis are Staphylococcus aureus, beta-haemolytic streptococci, and gram-negative bacteria such as Escherichia coli. Often, they are undetectable in the body because of their toxin's that cause the damage and not the organisms per se. However, these organisms would have left their imprint in the host in the form of their nucleic acid genomes which cannot be detected by any cultural methods. Furthermore, uncultivable bacteria such as Chlamydia pneumoniae and Mycoplasma pneumoniae may also cause sepsis; these cannot be rapidly detected by existing methods. A rapid clinical diagnosis of fatal conditions is a must in instituting the correct antibiotics, thus saving the lives of such patients. This may also cut down the unnecessary use of antibiotics which otherwise may help in the development of antimicrobial resistance.

The existing gold standard in the diagnosis of sepsis is the “Blood Culture Technique”, which is the growth of viable microorganisms present in blood using an enriched liquid medium (i.e., broth culture) followed by sub culturing the positive cultures on solid culture media. This method requires 2-7 days of incubation for bacterial growth. The current guidelines recommend the collection of two or three blood culture sets for each sepsis episode observed in a patient. Hence, 10-15 mL of blood is collected per set and distributed evenly into two bottles, one for aerobic and another for anaerobic incubation. Such a large amount of blood may not be available in most cases, especially from new-born babies. As mentioned, the blood culture systems require about 100 mL of blood (20-30 mL×3 Sets) for diagnosis which is a limiting factor in neonatal or paediatric patients.

Furthermore, the cultivation of slow-growing organisms such as anaerobes require long term incubation and this may delay the identification of the etiological agents. Also, sufficient microbial load and cultivable bacteria are required for getting positive blood culture reports within the stipulated incubation time (2-7 days). This cannot be correlated to the existing scenario as trace amounts of etiological agents such as live or dead microorganisms and their cellular components cause the onset of sepsis. Hence, several such factors reduce the overall sensitivity of blood cultures and increase the turn.

Accordingly, there remains a need for a method to rapidly identify one or more microorganisms that cause sepsis from a sample.

### SUMMARY

In view of the foregoing, an embodiment herein provides a method of identifying a plurality of microorganisms from a sample using NAT. The method includes (i) extracting nucleic acid from a sample; (ii) preparing molecular biomarkers comprising a forward primer and a reverse primer for target genes of each of the plurality of microorganisms as a master mix; (iii) adding the extracted nucleic acid into the master mix to obtain a reaction mixture; (iv) performing a polymerase chain reaction (PCR) on the reaction mixture to amplify a region of interest of the extracted nucleic acid; (v) performing amplification for the amplicons by increasing a temperature of the reaction mixture from 65° C. to 99° C. in 0.1° C. increments every 2 to 5 seconds and simultaneously measuring fluorescence (Melt Temperature); (vi) generating a melt curve for each target gene based on the identified melt temperatures and an amplification curve after the PCR amplification; and (vii) determining, using a Melt Curve analysis technique, Ct values, endpoint fluorescence level and melting profiles of the target genes by analyzing the amplification and melt curves to identify the plurality of microorganism present in the sample.

In some embodiments, the PCR includes the steps of (i) reverse transcriptase at 50° C. for 15 minutes initially (ii) denaturating the reaction mixture for 5 minutes at 95° C. and then for 10 seconds at 95° C. to obtain single-stranded DNA to enable primer annealing, (iii) annealing the denatured reaction mixture for 30 seconds at 55° C. to enable the forward and reverse primers to bind to a complementary sequence of the single-stranded DNA, and (iv) performing extension step for 10 seconds at 72° C. to enable the Taq polymerase to bind to the 3′-end (three prime end) of the forward and reverse primers and extend the sequence, wherein the above three-cycle is repeated for 40 times to amplify the region of interest of the single-stranded DNA.

In some embodiments, the sample is pre-treated to remove the background of host nucleic acid, thereby increasing the reliability of the molecular analysis of the microorganisms in the sample.

In some embodiments, the nucleic acid is extracted from the sample by: (i) adding 20 μL (microlitre) ProteinaseK into the bottom of a microcentrifuge tube; (ii) adding up to 200 μL of the sample to the microcentrifuge tube; (iii) adding 200 μL lysis buffer to the sample, and mix the sample using a pulse-vortex for 15 seconds; (iv) incubating the sample in a 56° C. for 10 minutes; (v) centrifuging the microcentrifuge tube comprising the sample to remove drops from the inside of a lid; (vi) adding 200 μL of 96-100% ethanol to the sample, and mix again using the pulse-vortex for 15 seconds to obtain a mixture; (vii) centrifuging the microcentrifuge tube comprising the mixture to remove drops from the inside of the lid; (viii) adding the mixture to a mini spin column without wetting the rim, and centrifuging at 8000 revolution per minute (RPM) for 1 minute in room temperature; (ix) placing the mini spin column comprising the mixture in a first collection tube; (x) adding 500 μL buffer AW1 to the mini spin column without wetting the rim and centrifuge at 8000 RPM for 1 minute in the room temperature; (xi) placing the mini spin column in a second collection tube; (xii) adding 500 μL Buffer AW2 to the mini spin column without wetting the rim and centrifuging at full speed of 14000 RPM for 3 minutes in the room temperature; (xiii) placing the mini spin column in a new microcentrifuge tube; (xiv) adding 60 μL of 0.1×TE buffer directly onto the membrane of the mini spin column and close the cap; (xv) incubating at room temperature of 15° C. to 25° C. for 1 minute; and (xvi) centrifuging at 8000 RPM for 1 minute at the room temperature.

In some embodiments, if the volume of the sample is less than 200 μL for extracting the nucleic acid, the Phosphate-buffered saline (PBS) is added to bring the volume to up to 200 μL. In some embodiments, the clinical sample is a blood, pus, urine, cerebrospinal fluid, or other body exudates.

In some embodiments, the target genes are IC, V1, V3, Gram-Positive Bacteria, Gram-Negative Bacteria, Other Microbial Targets, and Antibiotic-Resistant Gene Markers such as Vancomycin Resistance, AMR-CARBAPENEMASE, AMR-ESBL, AMR-CTX-M, AMR-AMP C and AMR-Colistin Resistant Gene Markers. In some embodiments, 20 μL of the reaction mixture comprises 10 μL of the extracted nucleic acid, 1× PCR DNA Master Eva Green I, 0.7 μM of primers of respective targets genes and 40 μM of primers for RNAse P.

The method described herein is cost-effective, provides rapid turn-around time, high sensitivity/specificity, and rules out cross-reactivity and promised clinical validation. This method of identifying a plurality of microorganisms from the clinical samples does not require the cultivation of microorganisms from the sample. This method consists of a panel of molecular biomarkers that may be used with melt-analysis based PCR for the rapid identification of 37 species of microorganism and 32 antibiotic resistance biomarkers from a variety of samples (e.g., blood, pus, urine, cerebrospinal fluid, and other body exudates). The method identifies DNA and/or RNA, which are a part of the live or dead microorganism; their presence indicates the presence of the respective microbe in the sample.

This NAT-based method is a rapid, specific, and reliable technique for the detection of DNA/RNA of 37 species of microorganism and 35 antibiotic resistance biomarkers within 3 hours of sample collection. The method can be incorporated as an ICU related protocol and expedites treatment time. The clinicians can initiate patient treatment using antibiotics to which the pathogen is susceptible, thus reducing the mortality and morbidity due to MDR pathogens. Moreover, our nucleic acid-based rapid method that requires only 1 mL of blood gives a diagnosis of the clinical condition by identification of the nucleic acids of the etiological agents in 3-4 hours.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIGS. 1A & 1B illustrate a method for identifying a plurality of microorganisms from a sample using Nucleic Acid amplification Technology (NAT) according to an embodiment herein; and

FIGS. 2A-2E illustrate the analysis of melt curve graphs of a plurality of microorganisms from different samples according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted to not unnecessarily obscure the embodiments herein. The examples used herein are intended merely to facilitate an understanding of ways in which the embodiments herein may be practised and to further enable those of skill in the art to practice the embodiments herein. Accordingly, the examples should not be construed as limiting the scope of the embodiments herein.

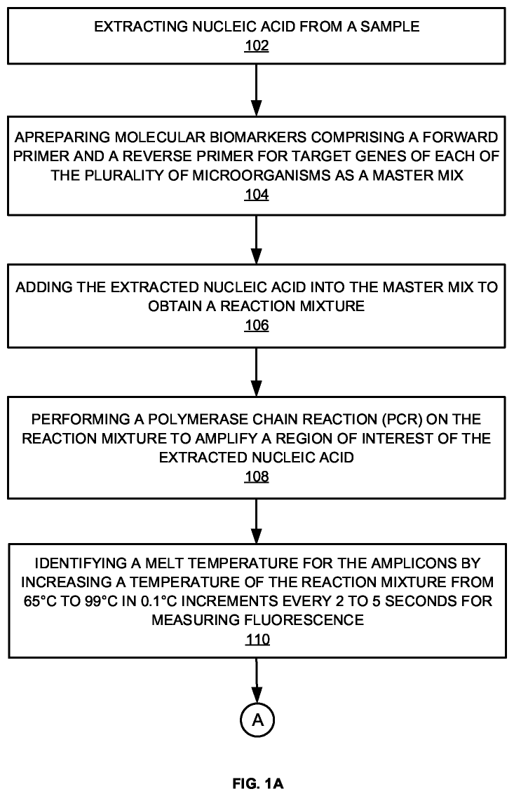

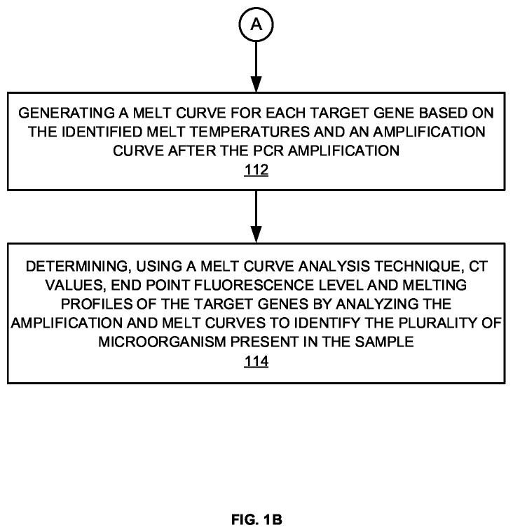

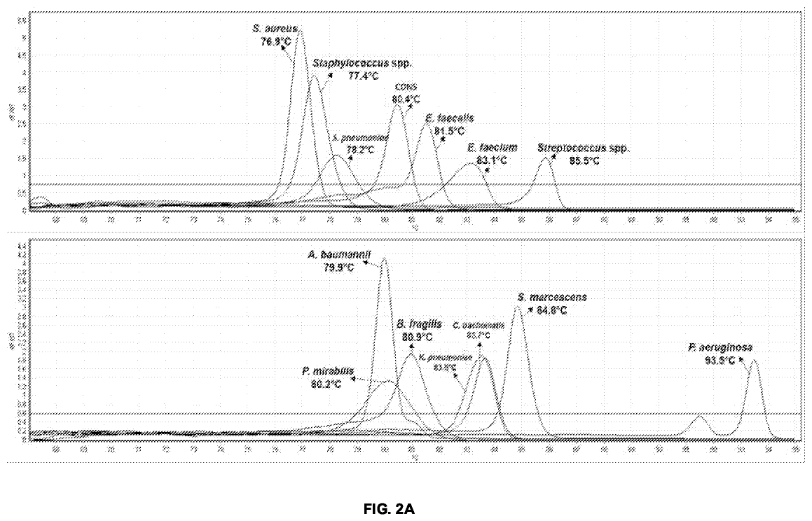

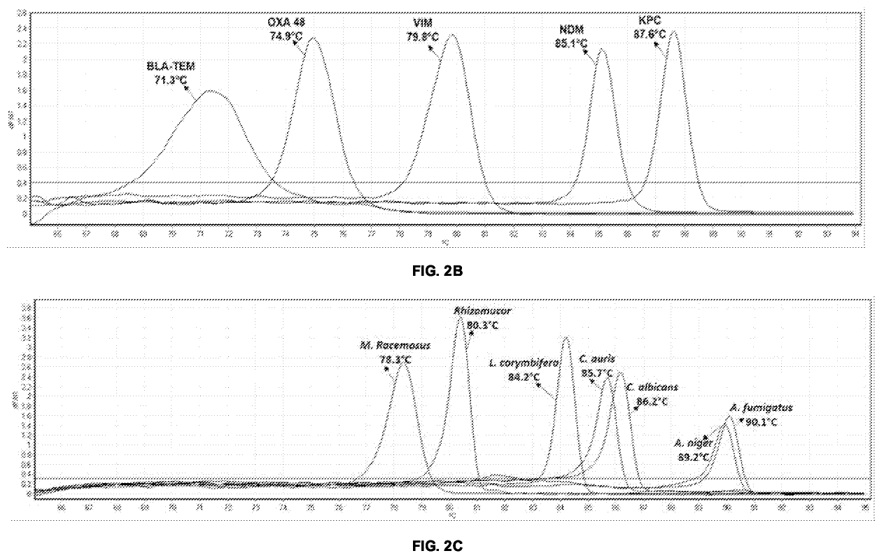

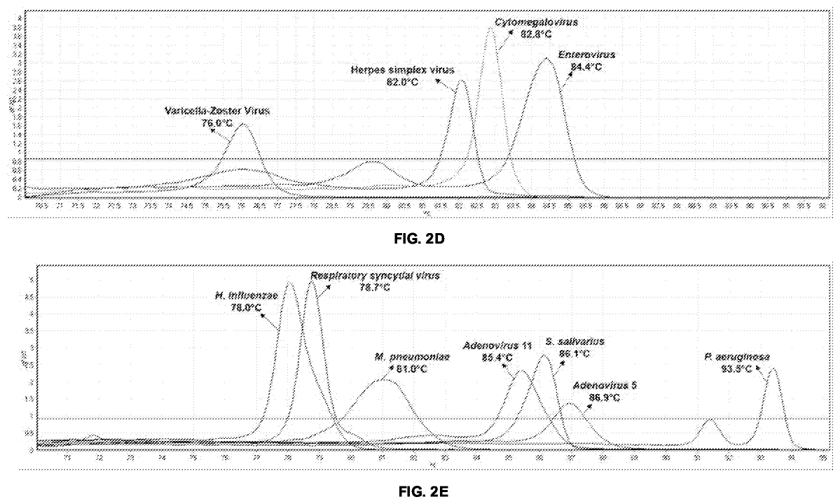

As mentioned, there remains a need for a rapid method of identifying one or more microorganisms from a sample using Nucleic Acid amplification Technology (NAT). Various embodiments disclosed herein provide a method of identifying a plurality of microorganisms from a sample using NAT, which is rapid and does not require the cultivation of viable bacteria from the sample. Referring now to the drawings, and more particularly to FIGS. 1 through 2E, where similar reference characters denote corresponding features consistently throughout the figures, preferred embodiments are shown.

FIGS. 1A & 1B illustrate a method of identifying a plurality of microorganisms from a sample using NAT according to the embodiment given herein. At step 102, the nucleic acid is extracted from a sample. At step 104, molecular biomarkers are prepared that includes a forward primer and a reverse primer for target genes of each of the plurality of microorganisms as a master mix. At step 106, the extracted nucleic acid is added into the master mix to obtain a reaction mixture. At step 108, a polymerase chain reaction (PCR) is performed on the reaction mixture to amplify a region of interest of the extracted nucleic acid. At step 110, a melt temperature is identified for the amplicons by increasing the temperature of the reaction mixture from 65° C. to 99° C. in 0.1° C. increments every 2 to 5 seconds for measuring fluorescence. At step 112, a melt curve is generated for each target gene based on the identified melt temperatures and an amplification curve after the PCR amplification. At step 114, Ct values, endpoint fluorescence level and melting profiles of the target genes are determined, using a Melt Curve analysis technique, by analyzing the amplification and melt curves to identify the plurality of microorganisms present in the sample.

In some embodiments, the PCR includes steps of (i) reverse transcriptase at 50° C. for 15 minutes initially (ii) denaturating the reaction mixture for 5 minutes at 95° C. and then for seconds at 95° C. to obtain single-stranded DNA to enable primer annealing, (iii) annealing the denatured reaction mixture for 30 seconds at 55° C. to enable the forward and reverse primers to bind to a complementary sequence of the single-stranded DNA, and (iv) performing extension step for 10 seconds at 72° C. to enable the Taq polymerase to bind to the 3′-end (three prime end) of the forward and reverse primers and extend the sequence, wherein the above three-cycle is repeated for 40 times to amplify the region of interest of the single-stranded DNA.

In some embodiments, the sample is pre-treated to remove the background of host nucleic acid, thereby increasing the reliability of the molecular analysis of the microorganisms in the sample.

In some embodiments, the nucleic acid is extracted from the sample by: (i) adding 20 μL Proteinase K into the bottom of a microcentrifuge tube; (ii) adding up to 200 μL of the sample to the microcentrifuge tube; (iii) adding 200 μL lysis buffer to the sample, and mix the sample using a pulse-vortex for 15 seconds; (iv) incubating the sample in a 56° C. for 10 minutes; (v) centrifuging the microcentrifuge tube comprising the sample to remove drops from the inside of a lid; (vi) adding 200 μL of 96-100% ethanol to the sample, and mix again using the pulse-vortex for 15 seconds to obtain a mixture; (vii) centrifuging the microcentrifuge tube comprising the mixture to remove drops from the inside of the lid; (viii) adding the mixture to a mini spin column without wetting the rim, and centrifuging at 8000 Revolutions Per Minute (RPM) for 1 minute in room temperature; (ix) placing the mini spin column comprising the mixture in a first collection tube; (x) adding 500 μL buffer AW1 to the mini spin column without wetting the rim and centrifuge at 8000 RPM for 1 minute in the room temperature; (xi) placing the mini spin column in a second collection tube; (xii) adding 500 μL Buffer AW2 to the mini spin column without wetting the rim and centrifuging at full speed of 14000 RPM for 3 minutes in the room temperature; (xiii) placing the mini spin column in a new microcentrifuge tube; (xiv) adding 60 μL of 0.1× TE buffer directly onto the membrane of the mini spin column and close the cap; (xv) incubating at room temperature of 15 to 25° C. for 1 minute; and (xvi) centrifuging at 8000 RPM for 1 minute at room temperature.

In some embodiments, if the volume of the sample is less than 200 lit for extracting the nucleic acid, the Phosphate-buffered saline (PBS) is added to bring the volume up to 200 μL. In some embodiments, the sample is a blood, pus, urine, cerebrospinal fluid, or other body exudates.

In some embodiments, the target genes are IC, V1, V3, Gram-Positive Bacteria, Gram-Negative Bacteria, Other Microbial Targets, and Antibiotic-Resistant Gene Markers such as Vancomycin Resistance, AMR-CARBAPENEMASE, AMR-ESBL, AMR-CTX-M, AMR-AMP-C, and AMR-Colistin Resistant Gene Markers. In some embodiments, 20 μL of the reaction mixture comprises of 10 μL of the extracted nucleic acid, 1× PCR DNA Master Eva Green I, 0.7 μM of primers of respective targets genes and 40 μM of primers for RNAse P.

In some embodiments, the sample is whole blood with anticoagulants, a synovial fluid, a pleural fluid, a cerebrospinal fluid, ascites fluid, pus, a bronchoalveolar lavage, a nasal douche fluid or a urine sample.

In some embodiments, the target genes may be selected from the following:
1. IC (Internal Control—RNAse P Gene)

### Pan Bacterial DNA Detection

2. V1
3. V3
4. V1F+V3R

### Gram-Positive Bacteria

5. Staphylococcus spp.
6. Staphylococcus aureus
7. Coagulase Negative Staphylococcus (CONS)
8. Streptococcus spp.
9. Streptococcus pyogenes (GAS)
10. Streptococcus agalactiae (GBSC)
11. Streptococcus pneumoniae
12. Enterococcus faecalis (Sod efs)
13. Enterococcus faecium (Sod efm)

### Gram-Negative Bacteria

14. Enterobacteriaceae
15. Escherichia coli
16. Klebsiella pneumoniae
17. Enterobacter aerogenes
18. Shigella spp.
19. Salmonella spp.
20. Proteus mirabilis
21. Serratia marcescens
22. Pseudomonas aeruginosa
23. Actinobacteria baumannii
24. Listeria monocytogenes
25. Bacteroides fragilis
26. Chlamydia trachomatis

### Fungal Targets

27. Mucor racemosus
28. Rhizomucor
29. Candida albicans
30. Candida auris
31. Aspergillus flavus
32. Aspergillus fumigatus
33. Aspergillus niger
34. Lichtheimia corymbifera

### Vancomycin Resistance

35. Van A Gene
36. Van B Gene
37. Van C1 Gene
38. Van C2/C3 Gene

### AMR-ESBL

39. b1a _{TEM} (β-Lactamase TEM)— ESBL Resistant Gene Markers
40. Oxa 1, 4, 3 (Oxacillin)
41. B1a SHV
42. VEB
43. PER

### AMR-CTX-M

44. CTX M-1
45. CTX M-2
46. CTX M-8
47. CTX M-9
48. CTX M-25

### AMR-AMP C

49. DHA
50. CMY-1/MOX
51. CMY-2/Lact
52. CIT
53. ACC
54. EBC
55. FOX

### AMR-CARBAPENEMASE

56. KPC (Klebsiella pneumoniae Carbapenemase Gene Markers)
57. GES
58. Oxa 48
59. VIM (Metallo β-Lactamases)
60. NDM (1-8)
61. IMP
62. SPM

### AMR-Colistin Resistant Gene Markers

63. MRC 1
64. MRC 2
65. MRC 3

### Antibiotic-Resistant Gene Markers

66. MRSA (Methicillin-resistant Staphylococcus aureus)

### Viral Targets

67. Varicella-Zoster virus
68. Herpes Simplex Virus
69. Cytomegalovirus
70. Enterovirus
71. Respiratory Syncytial Virus
72. Adenovirus 11
73. Adenovirus 5

FIGS. 2A-2E illustrate the analysis of melt curve graphs of a plurality of microorganisms from different samples according to an embodiment herein. In some embodiments, the melt curve analysis is performed using American Type Culture Collection (ATCC) strains and Patient samples. After PCR amplification, the melt curve unique to each gene target is identified and reported. The melt curve analysis is standardized for the target genes using ATCC cultures and patient samples with known pathogens. In some embodiments, the melt curve analysis is standardized for several Pan-Bacterial, Pan-Fungal, Viral targets, and several Antibiotic Resistance Markers which are a part of sepsis panel is shown as an example in FIGS. 2A-2E. The melting temperature of bacterial targets (FIG. 2A) such as Staphylococcus aureus amplicon is 76.9° C., Staphylococcus spp. is 77.4° C., Streptococcus pneumoniae is 78.2° C., Coagulase-negative staphylococci (CoNS) is 80.4° C., Enterococcus faecalis is 81.5° C., Enterococcus faecium is 83.1° C., Streptococcus spp. is 85.5° C., Acinetobacter baumannii is 79.9° C., Proteus mirabilis is 80.2° C., Bacteroides fragilis is 80.9° C., Klebsiella pneumoniae is 83.5° C., Chlamydia trachomatis is 83.7° C., Serratia marcescens is 84.8° C. and Pseudomonas aeruginosa is 93.5° C. Similarly, for the antibiotic resistance gene (FIG. 2B) b1a_{TEM} (extended-spectrum β-lactamase gene) is 71.3° C., OXA-48 (Carbapenemase gene) is 74.9° C., VIM (metallo-beta lactamase gene) is 79.8° C., NDM (New Delhi metallo-β-lactamase-1) is 85.1° C., KPC (Klebsiella pneumoniae carbapenemase) is 87.6° C. Similarly, for fungal targets (FIG. 2C) Mucor racemosus is 78.3° C., Rhizomucor is 80.3° C., Lichtheimia corymbifera is 84.2° C., Candida auris is 85.7° C., Candida albicans is 86.2° C., Aspergillus niger is 89.2° C., Aspergillus fumigatus is 90.1° C. Similarly, for the viral targets (FIG. 2D & 2E) such as Varicella-Zoster virus is 76.0° C., Herpes Simplex Virus is 82.0° C., Cytomegalovirus is 82.8° C., Enterovirus is 84.4° C., Respiratory Syncytial Virus is 78.7° C., Adenovirus 11 is 85.4° C. and Adenovirus 5 is 86.9° C. Similarly, for bacterial targets (FIG. 2E) Haemophilus influenzae is 78.0° C. and Streptococcus salivarius is 86.1° C.

In some embodiments, the melt curve analysis uses target-specific primers alone without a probe. The melt curve analysis determines a unique melt curve of each target gene is identified and this is found to be repeatable for the isolates from a different patient sample. The detection of unique melt curve by a real-time mode not by conventional mode of using Gel Doc. In a single melt curve analysis, the combination of the bacterial and viral target is detected.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practised with modification within the scope of appended claims.

I/We claim:

1. A method of identifying a plurality of microorganisms from a sample using Nucleic Acid amplification Technology (NAT), comprising:
extracting nucleic acid from a sample;
preparing molecular biomarkers comprising a forward primer and a reverse primer for target genes of each of the plurality of microorganisms as a master mix;
adding the extracted nucleic acid into the master mix to obtain a reaction mixture;
performing a polymerase chain reaction (PCR) on the reaction mixture to amplify a region of interest of the extracted nucleic acid;
identifying a melt temperature for the amplicons by increasing the temperature of the reaction mixture from 65° C. to 99° C. in 0.1° C. increments every 2 to 5 seconds for measuring fluorescence;
generating a melt curve for each target gene based on the identified melt temperatures and an amplification curve after the PCR amplification; and
determining, using a Melt Curve analysis technique, Ct values, endpoint fluorescence level and melting profiles of the target genes by analyzing the amplification and melt curves to identify the plurality of microorganisms present in the sample.

2. The method as claimed in claim 1, wherein the polymerase chain reaction comprises the steps of
denaturating the reaction mixture initially for 5 minutes at 95° C. and then for 10 seconds at 95° C. to obtain single-stranded DNA to enable primer annealing;
annealing the denatured reaction mixture for 30 seconds at 55° C. to enable the forward and reverse primer binds to a complementary sequence of the single-stranded DNA; and
performing extension step for 10 seconds at 72° C. to enable the Taq polymerase to bind to the 3′ end of the forward and reverse primers and extend the sequence, wherein the above three-cycle is repeated for 40 times to amplify the region of interest of the single-stranded DNA.

3. The method as claimed in claim 1, wherein the sample is pre-treated to remove the background of host nucleic acid, thereby increasing the reliability of the molecular analysis of the microorganisms in the sample.

4. The method as claimed in claim 1, wherein the nucleic acid is extracted from the sample by:
adding 20 μl, ProteinaseK into the bottom of a microcentrifuge tube;
adding up to 200 μl, of the sample to the microcentrifuge tube;
adding 200 μl, lysis buffer to the sample, and mix the sample using a pulse-vortex for 15 seconds;
incubating the sample in a 56° C. water bath for 10 minutes;
centrifuging the microcentrifuge tube comprising the sample to remove drops from the inside of a lid;
adding 200 μl, of 96-100% ethanol to the sample, and mix again using the pulse-vortex for 15 seconds to obtain a mixture;
centrifuging the microcentrifuge tube comprising the mixture to remove drops from the inside of the lid;
adding the mixture to a mini spin column without wetting the rim, and centrifuging at 8000 Revolutions Per Minute (RPM) for 1 minute in room temperature;
placing the mini spin column comprising the mixture in a first collection tube;
adding 500 μL buffer AW1 to the mini spin column without wetting the rim and centrifuge at 8000 RPM for 1 minute at the room temperature;
placing the mini spin column in a second collection tube;
adding 500 μL Buffer AW2 to the mini spin column without wetting the rim and centrifuging at full speed of 14000 RPM for 3 minutes in the room temperature;
placing the mini spin column in a new microcentrifuge tube;
adding 60 μl, of 0.1×TE buffer directly onto the membrane of the mini spin column and close the cap;
incubating at room temperature of 15° C. to 25° C. for 1 minute; and
centrifuging at 8000 RPM for 1 minute at room temperature.

5. The method as claimed in claim 4, wherein if the volume of the sample is less than 200 μL, for extracting the nucleic acid, the Phosphate-buffered saline (PBS) is added to bring the volume up to 200 μL.

6. The method as claimed in claim 1, wherein the sample is a blood, pus, urine, cerebrospinal fluid, or other body exudates.

7. The method as claimed in claim 1, wherein the target genes are IC, V1, V3, Gram-Positive Bacteria, Gram-Negative Bacteria, Other Microbial Targets, Antibiotic-Resistant Gene Markers, Vancomycin Resistance, AMR-CARBAPENEMASE, AMR-ESBL, AMR-CTX-M, AMR-AMP-C and AMR-Colistin Resistant Gene Markers.
